(19) United States
(12) Patent Application Publication
(10) Pub. No.: US 2006/0205716 A1
(43) Pub. Date: Sep. 14, 2006

(54) Microbicidal agents

(76) Inventors: Peter Wachtler, Krefeld (DE); Martin Kugler, Leichlingen (DE)

Correspondence Address: Lanxess Corporation;Law & Intellectual Property Department, 111 Ride Park West Drive, Pittsburgh, PA, 15275-1112, US

(21) Appl. No.: 10/553,526

(22) PCT Filed: Apr. 14, 2004
(86) PCT No.: PCT/EP04/03911

§ 371 Date: Oct. 11, 2005

(30) Foreign Application Priority Data
Apr. 23, 2003 (DE) 103 18 366.3

(51) Int. Cl. A61K 31/54

(52) U.S. Cl. 514/227.2

(57) ABSTRACT

The invention relates to antimicrobial, synergistically active mixtures of p-chloro-m-cresol (CMC) and/or its sodium or potassium salt and 2-methyl-2H-isothiazol-3-one (MIT)

The present application provides novel mixtures comprising p-chloro-m-cresol (CMC) and 2-methyl-2H-isothiazol-3-one (MIT), processes for their preparation and their use for protecting industrial materials and products against attack and destruction by microorganisms, and also microbicidal compositions based on these novel mixtures.

p-Chloro-m-cresol (CMC) and its sodium or potassium salt are active compounds which have been used in practice for a long time to prepare microbicidally active formulations and disinfectants. In principle, these active compounds have broad antimicrobial action against microorganisms such as bacteria, fungi and yeasts and are distinguished in an advantageous manner by good chemical and thermal stability. However, owing to the relatively high lipophilicity of CMC, in some applications the efficacy is not always satisfactory, and as a consequence the required application concentrations may be in a range which is economically unfavourable.

2-Methyl-2H-isothiazol-3-one (MIT) is a biocidally active compound with pronounced antibacterial action and a chemical and thermal stability which is sufficient for many industrial applications. However, owing to the limited activity spectrum (preferentially bacteria) and the not always satisfactory speed for the onset of action of this active compound, the practical requirements for a comprehensibly applicable biocide product are not met.

Surprisingly, we have now found novel mixtures based on p-chloro-m-cresol and/or its sodium or potassium salt (CMC) and 2-methyl-2H-isothiazol-3-one (MIT) which overcome the disadvantages of the respective individual components in an advantageous manner and thus contribute to improving the prior art.

Accordingly, the present invention relates to mixtures which are characterized in that they comprise p-chloro-m-cresol (CMC) and/or its sodium or potassium salt and 2-methyl-2H-isothiazol-3-one (MIT) as active components.

The mixtures according to the invention are highly active against microorganisms and can be used for protecting industrial materials against attack and destruction by microorganisms.

In addition, surprisingly, the mixtures according to the invention are distinguished in that, in specific mixing ratios, they have an unexpectedly high, synergistic enhancement of activity. As a consequence, the concentrations required of the mixtures according to the invention for protecting industrial products can be reduced compared to the concentrations required of the respective individual compounds. This is extremely advantageous from an economical, ecological and technical point of view and contributes to increasing the preservation quality.

The active compound mixtures according to the invention may preferably be used to preserve functional fluids and aqueous industrial products susceptible to attack by microorganisms.

Applications for the following industrial materials and products may be mentioned by way of example, but not by way of limitation:
chemical products for the building industry, such as concrete additives based, for example, on molasses, lignosulfonates or polyacrylates, bitumen emulsions or sealants
printing thickeners based on natural products such as alginates, guar meals, gum arabic, maize, wheat or rice starches
auxiliaries for the leather, textile or photochemical industry
cooling lubricant concentrates and/or aqueous emulsions or dilutions thereof for metal processing based on mineral oil-containing, semisynthetic or synthetic concentrates
glues and adhesives based on known raw materials of animal, vegetable or synthetic origin
polymer dispersions based, for example, on polyacrylate, polystyrene acrylate, styrenebutadiene, polyvinyl acetate, inter alia
starch solutions or slurries or other starch-based products, such as, for example, printing thickeners or wallpaper paste
slurries of other raw materials, such as color pigments (for example iron oxide pigments, carbon black pigments, titanium dioxide pigments) or slurries of fillers and coating pigments, such as kaolin, calcium carbonate or talc
precursors and intermediates of the chemical industry, for example in the production and storage of dyestuffs
solvent-borne or water-borne inks
wax and clay emulsions

The mixtures according to the invention may additionally comprise one or more further biocidally active compounds or be combined with these by separate addition to the products to be protected. The compounds
1,2-benzisothiazol-3(2H)-one
butyl-, ethyl-, methyl-, propyl-paraben
1,2-dibromo-2,4-dicyanobutane (Tektamer 38)
2,2-dibromo-3-nitrilopropionamide (DBNPA)
2-bromo-2-nitropropane-1 3-diol (bronopol)
5-chloro-2-methyl-4-isothiazolin-3-one/2-methyl-4-isothiazolin-3-one
(CMIT/MIT)
benzalkonium chloride
benzyl alcohol
boric acid
bromochlorodimethylhydantoin
cetylpyridinium chloride
diazolidinylurea
dichlorobenzyl alcohol
didecyldimethylammonium chloride
dimethylolurea
ethylene glycol hemiformal
ethylene glycol bishemiformal
formaldehyde
imidazolidinylurea
iodopropargyl butylcarbamate (IPBC)
methylene bisthiocyanate
3,3-dimethylol-5,5-dimethylhydantoin
N-methylolurea
2-n-octylisothiazolin-3-one (OIT)
phenoxyethanol
phenylethyl alcohol
o-phenylphenol (OPP)
poly(hexamethylenebiguanide)hydrochloride
tetramethylolacetylenediurea (TMAD)
trimethylene-2-methylisothiazolinon-3-one (Promexal)
   may be mentioned as preferred co-components.

The amounts of p-chloro-m-cresol (CMC) and/or its sodium or potassium salt and 2-methyl-2H-isothiazol-3-one (MIT) in the mixtures according to the invention may be varied within a wide range. In mixtures with broad antimicrobial action which serve to protect functional fluids and aqueous industrial products, the ratio of p-chloro-m-cresol (CMC) and/or its sodium or potassium salt to 2-methyl-2H-isothiazol-3-one (MIT) is generally a weight ratio of from 100:1 to 1:100, preferably from 50:1 to 1:50, particularly preferably from 10:1 to 1:10.

In the protection of materials, the mixtures according to the invention are used for protecting industrial materials, in particular for protecting aqueous functional fluids and water-containing industrial products.

They are effective against microorganisms of the most different types, such as, for example, bacteria, moulds, yeasts and also slime organisms.

They following species may be mentioned by way of example, but not by way of limitation:

Bacteria:

Alcaligenes, such as Alcaligenes faecalis, Bacillus, such as Bacillus subtilis, Escherichia, such as Escherichia coli, Proteus, such as Proteus vulgaris, Pseudomonas, such as Pseudomonas aeruginosa or Pseudomonas fluorescens, Staphylococcus, such as Staphylococcus aureus.

Yeasts:

Candida, such as Candida albicans, Geotrichum, such as Geotrichum candidum, Rhodotorula, such as Rhodotorula rubra, Saccharomyces, such as Saccharomyces cerevisiae.

Fungi:

Alternaria, such as Alternaria tenuis, Aspergillus, such as Aspergillus niger, Chaetomium, such as Chaetomium globosum, Fusarium, such as Fusarium solani, Lentinus, such as Lentinus tigrinus, Paecilomyces, such as Paecilomyces variotti, Penicillium, such as Penicillium glaucum.

The mixtures according to the invention can be prepared by mixing the individual components with one another, if appropriate with addition of one or more solvents and if appropriate further antimicrobially active compounds.

Depending on their respective physical and/or chemical properties, the mixtures according to the invention can be applied either separately in the form of a metered addition of the individual active compounds, in which case the concentration ratio may be individually adjusted depending on the preservation problem present, or the finished active compound mixture may be metered in. For this, it is possible to convert the mixture according to the invention beforehand into a customary formulation, such as, for example, a solution, emulsion, suspension, powder, foams, pastes, granules, aerosols and microencapsulations in polymeric substances.

These formulations may be prepared in a manner known per se, for example by mixing the mixture according to the invention or the individual active compounds comprised therein with extenders, i.e. liquid solvents, pressurized liquefied gases and/or solid carriers, if appropriate with the use of surfactants, i.e. emulsifiers and/or dispersants and/or foam formers. If the extender used is water, it is also possible to use, for example, organic solvents as auxiliary solvents. Essentially, suitable liquid solvents include: alcohols, such as butanol or glycol, and also ethers and esters thereof, ketones, such as acetone, methyl ethyl ketone, methyl isobutyl ketone or cyclohexanone, strongly polar solvents, such as dimethylformamide, N-methylpyrrolidone or dimethyl sulfoxide, and also water; liquefied gaseous extenders or carriers are to be understood as meaning liquids which are gaseous at ambient temperature and under atmospheric pressure, for example aerosol propellants, such as halogenated hydrocarbons, and also butane, propane, nitrogen and carbon dioxide; suitable solid carriers are: for example ground natural minerals, such as kaolins, clays, talc, chalk, quartz, attapulgite, montmorillonite or diatomaceous earth, and ground synthetic minerals, such as finely divided silica, aluminum oxide and silicates; suitable solid carriers for granules are: for example crushed and fractionated natural rocks, such as calcite, marble, pumice, sepiolite and dolomite, and also synthetic granules of inorganic and organic meals, and granules of organic material such as sawdust, coconut shells, corn cobs and tobacco stalks; suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, and also protein hydrolysates; suitable dispersants are: for example lignosulfite waste liquors or polyacrylates.

Tackifiers and thickeners such as carboxymethylcellulose and natural and synthetic polymers in the form of powders, granules or latices, such as gum arabic, polyvinyl alcohol, polyvinyl acetate, and also natural phospholipids, such as cephalins and lecithins and synthetic phospholipids can be used in the formulations. Other possible additives are mineral and vegetable oils.

The present invention furthermore provides microbicidal compositions based on the mixtures according to the invention, which compositions comprise at least one solvent or diluent and also, if appropriate, processing auxiliaries and if appropriate further antimicrobially active compounds.

The microbicidal compositions or formulated concentrates used for protecting industrial materials comprise the active compounds p-chloro-m-cresol (CMC) and/or its sodium or potassium salt and 2-methyl-2H-isothiazol-3-one (MIT), calculated as the sum of both active compounds, in a concentration of from 5 to 80% by weight, preferably from 10 to 60% by weight.

The use concentrations of the active compound combinations to be used according to the invention depend on the nature and the occurrence of the microorganisms to be controlled, on the initial microbial load, on the expected storage time of the products to be protected and on the composition of the end products at risk from microbiological attack. The optimum amount to be employed can be determined by preliminary tests and test series on a laboratory scale and by supplementary operational tests. In general, the use concentrations are in the range from 0.01 to 5% by weight, preferably from 0.05 to 1.0% by weight, of the mixture according to the invention, based on the material to be protected.

The surprising enhancement of the activity of the mixtures according to the invention is documented in the examples below:

### EXAMPLES

There are certain germs which are particularly relevant in practice, such as, for example, Pseudomonas fluorescens (Example 1), where the mixtures according to the invention are notable for synergistic effects, i.e. the activity of the mixture is greater than the activity of the individual components.

The observed synergism of the mixtures according to the invention can be determined by the following mathematical approach (cf. F. C. Kull, P. C. Elisman, H. D. Sylwestrowicz and P. K. Mayer, Appl. Microbiol. 9, 538 (1961):

$$synergistic index ( SI ) = \frac{{Q}_{a}}{{Q}_{A}} + \frac{{Q}_{b}}{{Q}_{B}}$$

where
Qₐ=the amount of component A in the active compound mixture required to achieve the desired effect, i.e. no microbial growth,
Q_{A}=the amount of component A which, applied on its own, suppresses the growth of the microorganisms,
Q_{b}=the amount of component B in the active compound mixture which suppresses the growth of the microorganisms, and
Q_{b}=the amount of component B which, applied on its own, suppresses the growth of the microorganisms.

A synergistic index of SI<1, in accordance with the above formula, indicates a synergistic effect for the active compound mixture.

Using the calculations below, the synergistic enhancement of activity is documented by way of example, but not by way of limitation.

### Example 1

### Synergism of p-chloro-m-cresol (CMC)/2-methyl-2H-isothiazol-3-one (MIT)

The minimum inhibitory concentration of the active compound combinations listed in Table 1 were examined using the test germ Pseudomonas fluorescens

TABLE 1
Pseudomonas fluorescens

| | | SI |
|---|---|---|
| Amount of pure active compounds required to suppress | | |
| growth (in ppm) | | |
| A = CMC | 150 (Q_{A)} | |
| B = MIT | 5 (Q_{B)} | |
| Q_{a,} Q_{b =} proportional amounts in active compound mixtures | | |
| required to suppress growth (in ppm) | | |
| CMC/MIT (9:1) | (Q_{a) =} 31.5/(Q_{b)=} 3.5 | 0.91 |
| CMC/MIT (8:2) | (Q_{a) =} 16/(Q_{b)=} 4 | 0.90 |
| CMC/MIT (7:3) | (Q_{a) =} 7/(Q_{b)=} 3 | 0.64 |
| CMC/MIT (6:4) | (Q_{a) =} 6/(Q_{b)=} 4 | 0.84 |
| CMC/MIT (5:5) | (Q_{a) =} 2.5/(Q_{b)=} 2.5 | 0.51 |
| CMC/MIT (4:6) | (Q_{a) =} 2/(Q_{b)=} 3 | 0.61 |
| CMC/MIT (3:7) | (Q_{a) =} 1.5/(Q_{b)=} 3.5 | 0.71 |
| CMC/MIT (2:8) | (Q_{a) =} 1/(Q_{b)=} 4 | 0.80 |
| CMC/MIT (1:9) | (Q_{a) =} 0.5/(Q_{b)=} 4.5 | 0.90 |

(in brackets = weight ratios of the active compounds in the mixture)

At certain concentration ratios, the combinations according to the invention have pronounced synergistic effect.

### Example 2

Preservation of a Cooling Lubricant Emulsion

What was examined was the preserving action of CMC/MIT mixtures according to the invention in a cooling lubricant emulsion (5% mineral oil/emulsifier concentrate/95% water). To this end, biocide-free and perserved samples of a cooling lubricant emulsion were repeatedly, at weekly intervals, exposed to microbiological stress using the following microorganisms:

Bacteria:
Pseudomonas aeruginosa
Pseudomonas fluorescens
Pseudomonas oleovorans
Pseudomonas rubescens
Pseudomonas stutzeri
Alcaligenes faecalis
Citrobacter freundii
Corynebacterium sp

Yeasts:
Rhodotorula rubra

Moulds:
Acremonium strictum
Fusarium solani
Geotrichum candidum

In each case, the bacteria were added separately from the yeasts/moulds.

The maximum duration of the test was 10 weeks (=10 contaminations), if the following limits were not exceeded during the weekly determination of the number of germs:

bacteria<10⁶ CFU/g

yeasts/moulds<10³⁻⁴ CFU/g.

Results:

a) Test with CMC as single component concentration employed=0.15% CMC, based on cooling lubricant emulsion
   number of weeks without colonization:
   bacteria=3 weeks
   moulds=10 weeks
   yeasts=10 weeks
b) Test with MIT as single component concentration employed 0.01% MIT, based on cooling lubricant emulsion
   number of weeks without colonization:
   bacteria=8 weeks
   moulds=5 weeks
   yeasts=8 weeks
c) Test with CMC/MIT mixture according to the invention, with the following amounts of active compounds resulting in the cooling lubricant emulsion:
   CMC=0.1% and 0.01% MIT
   number of weeks without colonization:
   bacteria=10 weeks
   moulds=10 weeks
   yeasts=10 weeks

Even though the concentration is reduced (CMC) or identical (MIT), the addition of a CMC/MIT mixture according to the invention results in a total activity which is considerably improved compared to the individual active compounds, i.e. full activity against all microorganisms used for testing is maintained over a test period of 10 weeks.

1. A mixture of biocidally active compounds, characterized in that it comprises p-chloro-m-cresol (CMC) and/or its sodium or potassium salt and 2-methyl-2H-isothiazol-3-one (MIT).

2. The mixture according to claim 1, characterized in that the weight ratio of p-chloro-m-cresol (CMC) and/or its sodium or potassium salt to 2-methyl-2H-isothiazol-3-one (MIT) is from 100:1 to 1:100.

3. A microbicidal composition comprising an active compound mixture according to at least one of claims 1 and 2 and at least one auxiliary from the group of the solvents or solvent mixtures, extenders, surfactants and, if appropriate, further antimicrobial active compounds.

4. The use of a mixture according to at least one of claims 1 and 2 for protecting industrial materials against attack and/or destruction by microorganisms.

5. The use according to claim 4, characterized in that the industrial materials are aqueous industrial products and aqueous functional fluids.

6. A process for preparing a mixture according to claim 1, characterized in that the individual components are mixed, if appropriate with addition of solvents or solvent mixtures, extenders, surfactants and further antimicrobially active compounds.

7. A method of protecting industrial materials against attack and/or destruction by microorganisms, characterized in that the industrial materials are mixed or treated with a mixture according to at least one of claims 1 and 2.

8. A method of protecting industrial materials against attack and/or destruction by microorganisms, characterized in that the industrial materials are mixed or treated with the individual active compounds of the mixture according to claim 1, if appropriate with addition of solvents or solvent mixtures, extenders, surfactants and further antimicrobially active compounds.

9. Industrial material comprising an active compound mixture according to least one of claims 1 and 2.
